(19) United States
(12) Patent Application Publication
(10) Pub. No.: US 2024/0420806 A1
(43) Pub. Date: Dec. 19, 2024

(54) METHOD, APPARATUS, AND SYSTEM WITH PHYSICAL PROPERTY PREDICTION INFERENCE AND/OR TRAINING

(71) Applicants: Samsung Electronics Co., Ltd., Suwon-si (KR); NEW YORK UNIVERSITY, New York, NY (US)

(72) Inventors: Seokhwan HONG, Suwon-si (KR); Hogyeong KIM, Suwon-si (KR); Hoshik LEE, Suwon-si (KR); Kyunghyun CHO, New York, NY (US)

(73) Assignees: Samsung Electronics Co., Ltd., Suwon-si (KR); NEW YORK UNIVERSITY, New York, NY (US)

(21) Appl. No.: 18/678,836

(22) Filed: May 30, 2024

Related U.S. Application Data
(60) Provisional application No. 63/521,170, filed on Jun. 15, 2023.

(30) Foreign Application Priority Data
Aug. 24, 2023 (KR) 10-2023-0111447

(51) Int. Cl. G16C 20/30; G16C 20/70

(52) U.S. Cl. CPC G16C 20/30; G16C 20/70

(57) ABSTRACT

A method, apparatus, and system with physical property prediction inference and/or training is provided. A processor-implemented method includes predicting physical properties of a target material using a machine learning model provided an input that is based on a target feature vector, where the target feature vector corresponds to the target material, where the machine learning model is configured to predict the physical properties of the target material based on a multi-dimensional space that is dependent on feature vectors representing respective structures of materials and relation information between the materials.

### CROSS-REFERENCE TO RELATED APPLICATIONS

This application claims the benefit under 35 U.S.C. § 119(e) of U.S. Provisional Application No. 63/521,170 filed on Jun. 15, 2023, in the U.S. Patent and Trademark Office, and claims the benefit under 35 USC § 119(a) of Korean Patent Application No. 10-2023-0111447 filed on Aug. 24, 2023, in the Korean Intellectual Property Office, the entire disclosures, all of which, are incorporated herein by reference for all purposes.

### BACKGROUND

### 1. Field

The following description relates to a method, apparatus, and system with physical property prediction inference and/or training.

### 2. Description of Related Art

There are various types of machine learning models. For example, one type of machine learning model is a neural network. With the advancement of machine learning models, electronic devices in various fields may use the same to analyze, extract features from, and/or generate valuable information about information input to the machine learning model, as non-limiting examples.

Typically, to predict physical properties of materials numerous experiments are required to be conducted by many researchers. However, such researcher predicted physical properties based on researcher performed experiments may not have high accuracies. Even when a model trained using unlabeled molecular structures is used to help predict physical properties of a material it is challenging for the trained model to achieve a sufficiently high accuracy due to the significant property changes that occur in such materials, including property changes that occur due to minor changes in material structures of the material.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, a processor-implemented method includes predicting physical properties of a target material using a machine learning model provided an input that is based on a target feature vector, where the target feature vector corresponds to the target material, where the machine learning model is configured to predict the physical properties of the target material based on a multi-dimensional space that is dependent on feature vectors representing respective structures of materials and relation information between the materials.

Each of the feature vectors may correspond to a node of a knowledge graph as a material representation of a material structure or molecular structure of a corresponding material, and the relation information may correspond to an edge of the knowledge graph.

With respect to the multi-dimensional space, the relation information may correspond to changes in physical property values indicating changes in characteristics according to structural changes among the materials.

Each of the feature vectors may correspond to a node of a knowledge graph, and the relation information may correspond to an edge of the knowledge graph, and the changes in physical property values may be represented in the knowledge graph in a form of consecutive numbers and configured with multi-dimensional edges based on the materials.

Each of the feature vectors may correspond to a node of a knowledge graph, and the relation information may correspond to an edge of the knowledge graph, and the knowledge graph may have a triple structure having a head entity and a tail entity respectively corresponding to the nodes, and the relation information may be related to a change in a physical property value between the head entity and the tail entity.

Each of the feature vectors may correspond to a node of a knowledge graph, and the relation information may correspond to an edge of the knowledge graph, and the prediction machine learning model may be trained to predict physical properties corresponding to a distance between the nodes in a vector space, as the multi-dimensional space.

Each of the feature vectors may correspond to a node of a knowledge graph, and the relation information may correspond to an edge of the knowledge graph, the multi-dimensional space may be a vector space embedded with the relation information, and the method may further include training the machine learning model based on a first loss based on molecular contrastive learning of representations (MolCLR) and a weighted second loss corresponding to the knowledge graph.

In one general aspect, a non-transitory computer-readable storage media may store a machine learning model and instructions that, when executed by one or more processors, cause the one or more processors to perform any one, combination, or all operations or methods described herein.

In a general aspect, a processor-implemented method includes generating a knowledge graph having, as respective nodes of the knowledge graph, a head entity corresponding to a first feature vector representing a structure of a first material and a tail entity corresponding to a second feature vector representing a structure of a second material, and having relation information between the first material and the second material as an edge of the knowledge graph, embedding the knowledge graph into a vector space, and training a prediction machine learning model based on the relation information embedded in the vector space.

The first feature vector may correspond to a first molecular representation representing a molecular structure of the first material, the second feature vector may correspond to a second molecular representation representing a molecular structure of the second material, and the first feature vector and the second feature vector may be calculated based on molecular contrastive learning of representations (MolCLR).

The first molecular representation and the second molecular representation may respectively correspond to different but correlated molecular graphs.

The relation information may include changes in physical property values indicating changes in characteristics according to structural changes among the first and second materials.

The embedding of the knowledge graph into the vector space may include an embedding of the knowledge graph into the vector space using an embedding model configured to perform embedding based on a distance between a result of adding the relation information to the head entity and the tail entity.

The training of the prediction machine learning model may be based on respective outputs of a first neural network configured to output a first latent vector corresponding to a molecular structure of the head entity, and a second neural network configured to output a second latent vector corresponding to a molecular structure of the tail entity, and the training of the prediction machine learning model may include training, dependent on the respective outputs, a relation neural network configured to enable an embedding vector corresponding to a change in a physical property value between the first latent vector and the second latent vector to be matched to the relation information.

The training of the prediction machine learning model may include training the prediction machine learning model by combining the relation information with a respective unique structural characteristic of each of the first material and the second material.

The training of the prediction machine learning model may include training the prediction machine learning model to reflect a tendency of changes in physical property values among the respective nodes according to respective changes in structural characteristics of the first and second materials.

The training of the prediction machine learning model may include calculating a second loss corresponding to the knowledge graph based on a distance between the tail entity and the head entity translated by the relation information in the vector space, and training the prediction machine learning model based on a weighting of the second loss.

The calculating of the second loss may include calculating the second loss corresponding to the knowledge graph as a negative margin loss calculated from the embedding of the knowledge graph.

The training of the prediction machine learning model based on the weighted second loss may include training the prediction machine learning model based on a first loss based on molecular contrastive learning of representations (MolCLR) and the weighted second loss.

In one general aspect, an electronic apparatus includes one or more processors configured to execute instructions, and a memory storing the instructions, which when executed by the one or more processors, configures the one or more processors to predict physical properties of a target material using a machine learning model provided an input that is based on a target feature vector, where the target feature vector corresponds to the target material, where the machine learning model is configured to predict the physical properties of the target material based on a multi-dimensional space that is dependent on feature vectors representing respective structures of materials and relation information between the materials.

Other features and aspects will be apparent from the following detailed description, the drawings, and the claims.

### BRIEF DESCRIPTION OF THE DRAWINGS

FIG. 1 illustrates an example method of training a machine learning model to predict physical properties of materials according to one or more embodiments.

FIG. 2A illustrates an example knowledge graph according to one or more embodiments.

FIG. 2B illustrates example relation information of a molecule-based knowledge graph according to one or more embodiments.

FIG. 3 illustrates an example method of training a graph neural network (GNN) based on a molecule-based knowledge graph according to one or more embodiments.

FIG. 4 illustrates an example method of predicting physical properties using a trained neural network according to one or more embodiments.

FIG. 5 illustrates an example inference operation using a GNN, which has been trained based on a molecule-based knowledge graph, according to one or more embodiments.

FIG. 6 illustrates an example method of training a GNN when some data for training the GNN is missing in training data according to one or more embodiments.

FIG. 7 illustrates an example electronic apparatus (or system) according to one or more embodiments.

Throughout the drawings and the detailed description, unless otherwise described or provided, it may be understood that the same drawing reference numerals may refer to the same or like elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences within and/or of operations described herein are merely examples, and are not limited to those set forth herein, but may be changed as will be apparent after an understanding of the disclosure of this application, except for sequences within and/or of operations necessarily occurring in a certain order. As another example, the sequences of and/or within operations may be performed in parallel, except for at least a portion of sequences of and/or within operations necessarily occurring in an order, e.g., a certain order. Also, descriptions of features that are known after an understanding of the disclosure of this application may be omitted for increased clarity and conciseness.

The features described herein may be embodied in different forms, and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application. The use of the term “may” herein with respect to an example or embodiment (e.g., as to what an example or embodiment may include or implement) means that at least one example or embodiment exists where such a feature is included or implemented, while all examples are not limited thereto. The use of the terms “example” or “embodiment” herein have a same meaning (e.g., the phrasing “in one example” has a same meaning as “in one embodiment”, and “one or more examples” has a same meaning as “in one or more embodiments”).

Throughout the specification, when a component or element is described as being “on”, “connected to,” “coupled to,” or “joined to” another component, element, or layer it may be directly (e.g., in contact with the other component, element, or layer) “on”, “connected to,” “coupled to,” or “joined to” the other component, element, or layer or there may reasonably be one or more other components, elements, layers intervening therebetween. When a component, element, or layer is described as being “directly on”, “directly connected to,” “directly coupled to,” or “directly joined” to another component, element, or layer there can be no other components, elements, or layers intervening therebetween. Likewise, expressions, for example, “between” and “immediately between” and “adjacent to” and “immediately adjacent to” may also be construed as described in the foregoing.

Although terms such as “first,” “second,” and “third”, or A, B, (a), (b), and the like may be used herein to describe various members, components, regions, layers, or sections, these members, components, regions, layers, or sections are not to be limited by these terms. Each of these terminologies is not used to define an essence, order, or sequence of corresponding members, components, regions, layers, or sections, for example, but used merely to distinguish the corresponding members, components, regions, layers, or sections from other members, components, regions, layers, or sections. Thus, a first member, component, region, layer, or section referred to in the examples described herein may also be referred to as a second member, component, region, layer, or section without departing from the teachings of the examples.

The terminology used herein is for describing various examples only and is not to be used to limit the disclosure. The articles “a,” “an,” and “the” are intended to include the plural forms as well, unless the context clearly indicates otherwise. As non-limiting examples, terms “comprise” or “comprises,” “include” or “includes,” and “have” or “has” specify the presence of stated features, numbers, operations, members, elements, and/or combinations thereof, but do not preclude the presence or addition of one or more other features, numbers, operations, members, elements, and/or combinations thereof, or the alternate presence of an alternative stated features, numbers, operations, members, elements, and/or combinations thereof. Additionally, while one embodiment may set forth such terms “comprise” or “comprises,” “include” or “includes,” and “have” or “has” specify the presence of stated features, numbers, operations, members, elements, and/or combinations thereof, other embodiments may exist where one or more of the stated features, numbers, operations, members, elements, and/or combinations thereof are not present.

As used herein, the term “and/or” includes any one and any combination of any two or more of the associated listed items. The phrases “at least one of A, B, and C”, “at least one of A, B, or C”, and the like are intended to have disjunctive meanings, and these phrases “at least one of A, B, and C”, “at least one of A, B, or C”, and the like also include examples where there may be one or more of each of A, B, and/or C (e.g., any combination of one or more of each of A, B, and C), unless the corresponding description and embodiment necessitates such listings (e.g., “at least one of A, B, and C”) to be interpreted to have a conjunctive meaning.

Unless otherwise defined, all terms, including technical and scientific terms, used herein have the same meaning as commonly understood by one of ordinary skill in the art to which this disclosure pertains and specifically in the context on an understanding of the disclosure of the present application. Terms, such as those defined in commonly used dictionaries, are to be interpreted as having a meaning that is consistent with their meaning in the context of the relevant art and specifically in the context of the disclosure of the present application, and are not to be interpreted in an idealized or overly formal sense unless expressly so defined herein.

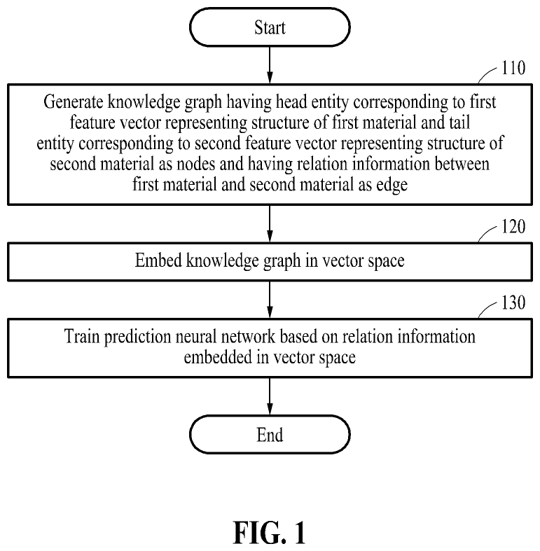

FIG. 1 illustrates an example method of training a machine learning model to predict physical properties of materials according to one or more embodiments. Respective operations in each of the various embodiments (e.g., descriptions of respective operations of each of FIGS. 1-7) described hereinafter may be performed sequentially, but not necessarily sequentially. For example, respective orders of the performance of the variously described operations with respect to each of FIGS. 1-7 may vary, and two or more of the variously described operations may be performed in parallel.

Referring to FIG. 1, an electronic apparatus (or system) may be configured to train (and/or perform inference operations using) a machine learning model to predict physical properties of materials. As a non-limiting example, the electronic apparatus may train a prediction neural network, as the machine learning model, through automated performances of operations 110 to 130 of FIG. 1.

In operation 110, the electronic apparatus may generate a knowledge graph having, as respective nodes, a head entity corresponding to a first feature vector representing the structure of a first material and a tail entity corresponding to a second feature vector representing the structure of a second material. The knowledge graph may include relation information between the first material and the second material as an edge. The first feature vector and the second feature vector may correspond to respective representations of materials, where the representations of the materials may each respectively include or represent a material structure or molecular structure of the materials. For example, the first feature vector may correspond to a first molecular representation of the molecular structure of the first material. The second feature vector may correspond to a second molecular representation of the molecular structure of the second material. As a non-limiting example, the first feature vector and the second feature vector may be respectively calculated based on, for example, molecular contrastive learning of representations (MolCLR). The first molecular representation and the second molecular representation may respectively correspond to different but correlated molecular graphs. The relation information may include information of or about changes in physical property values that indicate changes in characteristics according to structural changes among the materials. For example, the knowledge graph may have change values of characteristics (physical properties) among nodes as edges of the knowledge graph. An edge of the knowledge graph may indicate, for example, a continuous change in a physical property value or discontinuous change in a physical property value.

In one or more examples, the physical properties of a new material (e.g., a material for which physical properties may not be known) may be predicted using a trained prediction machine learning model. For example, the prediction machine learning model may be trained by generating a knowledge graph in which a representation of a material (e.g., a molecular representation) is defined as node(s) and training the prediction machine learning model based on the knowledge graph. For example, the prediction machine learning model may be trained to predict physical properties corresponding to the distances, in a vector space, between the nodes of the knowledge graph.

As a non-limiting example, the knowledge graph may be configured in a triple structure including three elements such as a head entity, relation information, and a tail entity. The head entity and the tail entity may correspond to various materials, such as a reference material or sample material, and may correspond to nodes of the knowledge graph. The relation information may represent a relation between the head entity and the tail entity and may correspond to an edge of the knowledge graph.

In an example, the knowledge graph may have two nodes that respectively correspond to the head entity and the tail entity, and an edge between the nodes representing the relation information. The head entity and the tail entity and the relation information may all be represented as vectors. In an example, the head entity and the tail entity may be represented as vectors, and the relation information of the edge may be represented as a matrix.

In operation 120, the electronic apparatus may embed the knowledge graph in the vector space by applying (e.g., inputting/providing) the knowledge graph generated in operation 110 to a graph neural network (GNN). In an example, the GNN may include a first neural network that outputs a first latent vector corresponding to the molecular structure of the head entity, a second neural network that outputs a second latent vector corresponding to the molecular structure of the tail entity, and a relation neural network that outputs relation information related to changes in physical property values between the first latent vector and the second latent vector. The vector space may be, for example, a Euclidean space, but examples are not limited thereto.

Embedding a knowledge graph in a vector space may mean embedding the components (e.g., a head entity and a tail entity corresponding to nodes, and relation information corresponding to an edge) of the knowledge graph into the vector space, and may be referred to as “knowledge graph embedding (KGE)”.

For example, the electronic apparatus may generate an arbitrary embedding vector from a normal distribution or Gaussian distribution when embedding entities and relation information in the vector space, or the electronic apparatus may generate an embedding vector using structural information of the knowledge graph. The electronic apparatus may map the elements of the knowledge graph into the vector space while maintaining the structural information of the knowledge graph. For example, deep learning or machine learning-based neural network models may typically internally use the representations of inputs rather than using the inputs as they are. The electronic apparatus may perform many downstream tasks using the representations of the knowledge graph by embedding the components of the knowledge graph into the vector space. Herein, as non-limiting examples, models trained by deep learning or machine learning will both be referred to as machine learning models.

The electronic apparatus may embed the difference between numerical labels in a multi-dimensional vector space regardless of the representations of nodes forming a graph in the form of a graph embedding algorithm. The electronic apparatus may embed all types of data that form a triple structure in a multi-dimensional space. The electronic apparatus may embed labels in the form of consecutive numbers, for example, between nodes in a triple structure of the knowledge graph in the multi-dimensional space.

In operation 130, the electronic apparatus may train the prediction machine learning model based on the relation information embedded in the vector space. The electronic apparatus may train the prediction machine learning model by combining the relation information embedded in the vector space with a unique structural characteristic of each of the first material and the second material. The electronic apparatus may train the prediction machine learning model such that the tendency of changes in physical property values among the nodes of the knowledge graph is reflected in the prediction machine learning model according to respective changes in structural characteristics of respective materials represented by each of the nodes. Herein, the “materials” may be understood to encompass both organic and inorganic matters. The “tendency of changes in physical property values” may be interpreted as the tendency (or degree) of changes in physical properties according to changes in structural characteristics of materials.

The electronic apparatus may train the prediction machine learning model on the changes in physical property values such that the distance between the tail entity and the head entity translated by the relation information is reflected in the vector space.

More particularly, the electronic apparatus may calculate a second loss corresponding to the knowledge graph based on the distance between the tail entity and the head entity translated by the relation information in the vector space. The electronic apparatus may train the prediction machine learning model based on a weighted second loss. The electronic apparatus may calculate the second loss corresponding to the knowledge graph as a negative margin loss calculated from the embedding of the knowledge graph. The negative margin loss may correspond to a kind of loss function that may involve calculations such that the difference between a head entity and a tail entity is closer to a relation vector corresponding to changes in physical property values during a knowledge graph learning process. When the negative margin loss is used, it may be possible to increase or maximize training efficiency because negative sampling may help classify the most similar things and the most different things during the learning process.

The electronic apparatus may train the prediction machine learning model based on training data, and a corresponding first loss based on MolCLR and the corresponding weighted second loss respectively of the prediction machine learning model. The electronic apparatus may improve the prediction accuracy of the prediction machine learning model for an inference, i.e., a prediction using a trained prediction neural network, of the physical properties of a target material by training the prediction machine learning model simultaneously with respect to the unique structural characteristics of materials and with respect to the changes in physical properties according to the structural changes of the materials. While an example knowledge graph with two or more nodes, which respectively represent different materials, and change information (e.g., relation information) of changes in physical properties between the different materials and the embedding of the vector space with respect to the same are discussed below, these explanations of the example knowledge graph and example embedding of the vector space are provided only for convenience of explanation and the same explanations are also applicable to training data that includes many other knowledge graphs, covering various materials and corresponding change information, and iterative training of the prediction machine learning model by embedding the same into a vector space of the prediction machine learning model.

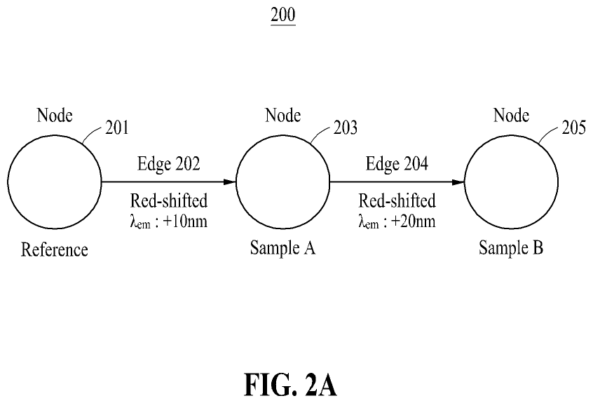

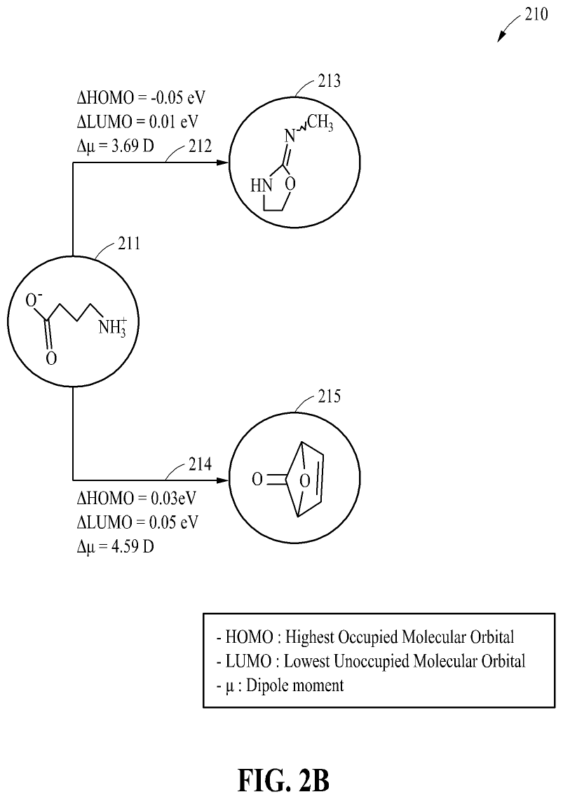

Accordingly, FIG. 2A illustrates an example knowledge graph according to one or more embodiments. For example, FIG. 2A illustrates a scheme of configuring a knowledge graph 200 including nodes (e.g., a first node 201, a second node 203, and a third node 205) and edges (e.g., a first edge 202 and a second edge 204).

In an example, the electronic apparatus (or system) may configure the knowledge graph 200 and perform a corresponding embedding operation to embed information of the knowledge graph 200 (e.g., only with the features of a node through a relationship with a neighboring node) into a vector space. Using a knowledge graph to obtain coordinates for determining a node embedding in a vector space may adjust the coordinates based on the relationships between a plurality of nodes. As a result, even when there are missing (e.g., not available) labels in the training data, a decrease in prediction performance using this training data may be relatively small.

The knowledge graph 200 may have, for example, a triple structure including a head entity, relation information, and a tail entity. The head entity and the tail entity may correspond to various materials, such as a reference material or sample material, and may be represented as the nodes (e.g., 201, 203, and 205). The relation information may represent a relation between nodes, for example, a relation between the head entity and the tail entity, and may be represented as the edges (e.g., 202 and 204).

The knowledge graph 200 may include, for example, the first node 201 corresponding to a reference material, the second node 203 corresponding to sample A, the third node 205 corresponding to sample B, the first edge 202 representing the relation between the first node 201 and the second node 203, and the second edge 204 representing the relation between the second node 203 and the third node 205. For example, material information corresponding to the nodes (e.g., 201, 203, and 205) of the knowledge graph and relation information corresponding to the edges (e.g., 202 and 204) may all be represented as vectors. Alternatively, the material information corresponding to the nodes (e.g., nodes 201, 203, and 205) of the knowledge graph may be represented as a vector, and the relation information corresponding to the edges (e.g., 202 and 204) may be represented as a matrix.

FIG. 2B illustrates an example relation information of a molecule-based knowledge graph according to one or more embodiments. FIG. 2B includes a molecule-based knowledge graph 210 that includes examples of relation information.

Example nodes 211, 213, and 215 of the knowledge graph 210 may represent feature vectors, and each of the feature vectors corresponds to a representation (e.g., molecular representation) of a material. An edge 212 may represent relation information one between the node 211 and the node 213, and an edge 214 may represent relation information two between the node 211 and the node 215.

The relation information one and/or the relation information two may represent changes in physical properties among material representations respectively corresponding to the nodes 211, 213, and 215. As a non-limiting example, each of the relation information one and/or the relation information two may respectively represent (or include) information of the highest occupied molecular orbital (ΔHOMO), the lowest unoccupied molecular orbital (ΔLUMO), and a dipole moment (Δμ), but examples are not limited thereto. The HOMO may correspond to an orbital having the highest energy level among orbitals of an atomic shell positioned one layer below an orbital having the LUMO. The LUMO is an orbital having the lowest energy among orbitals not filled with electrons and may correspond to an anti-bonding orbital. In other words, the LUMO is one layer above the HOMO and has a higher energy level than the HOMO. The gap between the LUMO and the HOMO may be referred to as “gap energy”. Due to gap energy, chemical side reactions may occur to generate solid electrolyte interphase (SEI). SEI may be understood as the formation of a solid membrane that acts as a thin separator through a chemical side reaction between electrolyte additives and lithium ions. In other words, a process that causes the above-described chemical side reaction may occur due to the LUMO and the HOMO. The dipole moment (μ) may correspond to a physical quantity representing the polarity of a system including two opposite electric charges. The magnitude of the dipole moment (μ) is equal to the value obtained by multiplying the magnitude (q) of a separated electric charge by the distance (r) between the electric charges, and may be a vector having a direction from a negative charge to a positive charge, for example. In chemistry, a dipole moment may correspond to a physical quantity that may explain the distribution of electrons between two bonded atoms. In other words, it may be possible to distinguish between polar and non-polar bonds using the dipole moment. A molecule having a net dipole moment, that is, a molecule having a net dipole moment other than zero, may correspond to a polar molecule. A molecule having a net dipole moment close to zero or equal to zero may correspond to a non-polar molecule. Relation information may further include various physical properties, such as melting points, boiling points, freezing points, and the like. As illustrated in FIG. 2A, “Red-shifted” written beneath each of the edges may correspond to a corresponding label that may be included in the training data.

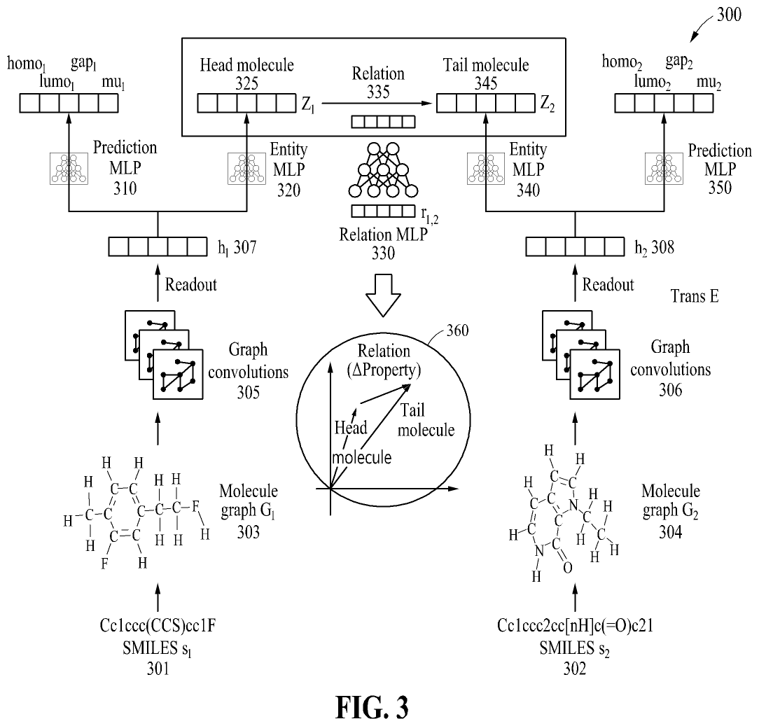

FIG. 3 illustrates an example method of training a graph neural network (GNN) based on a molecule-based knowledge graph according to one or more embodiments. FIG. 3 is a diagram 300 illustrating a process in which an electronic apparatus (or system) generates a knowledge graph from a simplified molecular input line entry system (hereinafter, “SMILES”) and trains a prediction neural network such that the distance between node embeddings of the knowledge graph is reflected in a vector space 360.

SMILES may correspond to a notation that represents a molecular structure in the form of strings that can be used to represent a molecular structure, such as for use in various machine learning/deep learning algorithms of a prediction machine learning model. In one example, the machine learning/deep learning algorithms may include those used for training a machine learning model with respect to natural language processing (NLP) for an input (or provided) molecular structure to the machine learning model for prediction of physical properties of the molecular structure. Elements included in the SMILES notation may be largely divided into five categories: atom, bond, ring, aromaticity, and branch.

In SMILES, an atom may be represented by a corresponding element symbol. For example, carbon may be denoted as C, nitrogen as N, oxygen as O, and chlorine as Cl. A bond may be represented using one of the eight symbols, for example, ·, —, =, #, $, :, /, \. A single bond may be represented using “—” and may be mostly omitted. Double, triple, and quadruple bonds are represented by the symbols “=”, “#”, and “$”, respectively. A ring may be written by breaking a bond at an arbitrary point and adding numbers to two atoms at the broken point. Aromaticity may include an aromatic ring formed when a carbon compound forms bonds in a planar ring shape to have a stable structure. An aromatic ring may be expressed in the same way as the ring described above, but atoms such as B, C, N, O, P, and S included in an aromatic ring may be expressed in lowercase letters. A branch means a branch of a molecule and may be expressed within parentheses “( )”. The first atom enclosed within parentheses and the first atom that comes out after the closing parenthesis may be connected to the same atom.

For convenience of explanation, while FIG. 3 illustrates character strings according to the SMILES notation that may be input to the electronic apparatus or otherwise obtained by (or read from a memory of) the electronic apparatus, examples are not limited thereto. In addition, or as an alternative, to the SMILES notation, various string-based representations or inputs in various data formats may be used to represent the molecular structure of a material for the training of, or for input to the trained prediction neural network in an inference operation, by electronic apparatus.

In an example, molecular representations, such as SMILES s₁ 301 and SMILES s₂ 302, may be input to (or otherwise obtained by) the electronic apparatus. The molecular representations SMILES s₁ 301 and SMILES s₂ 302 may be, for example, selected from among N mini-batches of molecular data within a training data used to train the prediction neural network.

With respect to SMILES s₁ 301 and SMILES s₂ 302, the electronic apparatus may extract a molecular graph G₁ 303 from s₁ 301 and extract a molecular graph G₂ 304 from s₂ 302. The electronic apparatus may translate the molecular representations (e.g., s₁ 301 and s₂ 302) into two different but correlated molecular graphs (e.g., G₁ 303 and G₂ 304) using a molecular graph augmentation strategy. An augmented molecular graph of the same molecule may be represented as a positive pair, while a molecular graph of different molecules may be represented as a negative pair.

A molecular graph may be defined as, for example, G=(V, E), where V and E may correspond to a node (atom) and an edge (chemical bond), respectively.

The electronic apparatus may obtain a molecular representation h₁ 307 by inputting the molecular graph G₁ 303 to a graph convolutional layer 305 of the GNN, where the output (or readout) of the graph convolutional layer 305 may correspond to the molecular representation h₁ 307. In addition, the electronic apparatus may obtain a molecular representation h₂ 308 by inputting the molecular graph G₂ 304 to a graph convolutional layer 306 of the GNN, where the output (or readout) of the graph convolutional layer 306 may correspond to the molecular representation h₂ 308. Each of the graph convolutional layer 305 and the graph convolutional layer 306 may include one or more convolution layers.

The GNN may perform a neighbor aggregation task that repeatedly updates a node representation. The aggregation update rule for the node function in the k-th layer of the GNN may be expressed by Equation 1 below.

$${a}_{v}^{( k )} = {AGGREGATE}^{( k )} ( \{ {h}_{u}^{( k - 1 )} : 𝓊 ∈ 𝒩 ( v ) \} ) , {h}_{v}^{( k )} = {COMBINE}^{( k )} ( {h}_{v}^{( k - 1 )} , {a}_{v}^{( k )} ) , \qquad Equation 1$$

Here, hᵥ^{(k)} may represent or denote a feature of node v in the k-th layer. hᵥ⁽⁰⁾ may be initialized by a node feature xᵥ. [symbol] (v) may represent or denote the set of all neighbors of node v. To additionally extract a graph-level feature h_{G}, a readout task may integrate the features of all nodes hᵤ^{(k)} in graph G given in Equation 2 below.

Equation 2:

$${h}_{G} = READOUT ( \{ {h}_{𝓊}^{( k )} : v ∈ G \} )$$

The prediction neural network may include, for example, a feature extractor f(·) and/or a nonlinear projection head g(·). The feature extractor may be modeled by the GNN. The feature extractor may map the molecular graphs to molecular representations. The feature extractor may map the molecular graphs to molecular representations using, for example, average pooling. The nonlinear projection head g(·) may be modeled by prediction multi-layer perceptrons (MLPs) 310 and 350, each with one hidden layer, as a non-limiting example.

An entity MLP 320 may map the molecular expression h1 307 to a latent vector Z1 325. The latent vector Z1 325 may correspond to a head molecule. The entity MLP 320 may output a first latent vector (e.g., the latent vector Z1 325) corresponding to the molecular structure of a head entity. The latent vector Z1 325 may correspond to a first feature vector (e.g., the molecular representation h1 307) representing the structure of a first material (e.g., the molecular representation SMILES s1 301).

An entity MLP 340 may map the molecular representation h2 308 to a latent vector Z2 345. The latent vector Z2 345 may correspond to a tail molecule. The entity MLP 340 may output a second latent vector (e.g., the latent vector Z2 345) corresponding to the molecular structure of a tail entity. The latent vector Z2 345 may correspond to a second feature vector (e.g., the molecular expression h2 308) representing the structure of a second material (e.g., the molecular representation SMILES s2 302).

The electronic apparatus may generate a knowledge graph having a head entity corresponding to the latent vector Z1 325 and a tail entity corresponding to the latent vector Z2 345 as nodes and having relation information 335 between the latent vector Z1 325 and the latent vector Z2 345 as an edge. Changes in molecular structures between the head entity and the tail entity may correspond to changes in physical properties, and therefore, the electronic apparatus may train the GNN based on changes in property values that indicate changes in characteristics according to (molecular) structural changes of the nodes.

Thus, the electronic apparatus may embed the knowledge graph in the vector space 360. For example, the electronic apparatus may embed the knowledge graph in the vector space using an embedding model (e.g., a TransE model) that performs embedding based on the distance between a result of adding the relation information 335 to the head entity corresponding to the latent vector Z1 325 and the tail entity corresponding to the latent vector Z2 345. For example, the electronic apparatus may embed the knowledge graph in the vector space using a relation neural network (e.g., a relation MLP 330) that enables an embedding vector corresponding to a change in a physical property value between the latent vector Z1 325 corresponding to the molecular structure of the head entity and the latent vector Z2 345 corresponding to the molecular structure of the tail entity to be matched to the relation information.

The electronic apparatus may derive node embeddings of the head molecule and the tail molecule using a pretrained model (e.g., MolCLR). The electronic apparatus may embed the head entity, the tail entity, and the relation information using a translation-based graph embedding model to learn a head-to-tail relation from each of the node embeddings.

The electronic apparatus may derive a second loss corresponding to the knowledge graph by calculating a negative margin loss as expressed by Equation 3 below from the embeddings of the head entity, the tail entity, and the relation information within a batch of training data.

$${ℒ}_{K G} = min ‖ {v}_{head} + {r}_{h e a d , t a i l} - {v}_{t a i l} ‖ \qquad Equation 3$$

Here, [symbol] _{head} may represent a vector corresponding to the head entity, [symbol] ₜₐᵢₗ may represent a vector corresponding to the tail entity, and r_{head,tail} may represent a relation vector (or matrix) between the head entity and the tail entity.

The electronic apparatus may train the prediction neural network (e.g., each of the prediction MLP 310 and the prediction MLP 350) by adjusting a weight of the second loss.

More particularly, the electronic apparatus may use MolCLR through the GNN, which may be a self-supervised learning framework. MolCLR may refer to an unsupervised learning technique that represents a molecule as a molecular graph to learn similar molecules and different molecules from an unlabeled dataset.

The MolCLR may be pre-trained through the GNN, differentiable representations may be learned by building molecular graphs and developing graph-neural-network encoders.

The GNN may have the molecular representation h1 307 and the molecular representation h2 308 as nodes and may have a network structure including a graph that represents relation information between the nodes (e.g., h1 307 and h2 308) through changes in physical property values.

The changes in physical property values corresponding to the relation information may exist in the form of numbers, and the prediction neural network may be trained on the changes in physical property values such that the distances between node embeddings in the vector space (e.g., Euclidean space) are reflected. In various examples, the electronic apparatus may show performance improvement on an open dataset, QM series, using the prediction neural network trained through the above-described process.

A node embedding for the GNN may be performed by, for example, a KGE model based on a translation distance method. The KGE model may represent entities and relation information using a distance-based scoring function. KGE models may treat relation information as a single operation. Herein, a KGE model may be referred to simply as an “embedding model”.

In an example, a head entity and a tail entity corresponding to the nodes of a knowledge graph and relation information corresponding to an edge may all be represented as vectors. In this example, the electronic apparatus may translate the head entity vector using the relation vector and then calculate the distance between the head entity vector and the tail entity vector by a score function. The score calculated by the score function may correspond to the above-described representation (e.g., the molecular representation).

In an example, entities (e.g., respective information of each of the nodes) and relation information may be embedded in a vector space using a TransE model among various embedding models based on the translation distance method. The TransE model may perform embedding such that the distance between a result of adding a relation vector (r) to a head entity vector (h) and a tail entity vector (t) is minimized. In other words, the TransE model may perform embedding such that the result of adding the relation vector (r) to the head entity vector (h) is as close as possible to the tail entity vector (t).

The head entity vector (h), the tail entity vector (t), and the relation vector (r) may be represented as vectors in the same d-dimensional space. Also, the score function may be, for example, fᵣ(h,t)=−∥h+r−t∥_{1/2}. The negative sign on the right-hand side of the score function may indicate that in the score function, as the distance (difference) between the result (h+r) of adding the relation vector (r) to the head entity vector (h) and the tail entity vector (t) increases, the score decreases.

In order to train the embedding model, the electronic apparatus may extract a triple structure from the knowledge graph. The electronic apparatus may train the embedding model using a high score when the extracted triple matches the knowledge graph. The electronic apparatus may train the embedding model using a low score when the extracted triple does not match the knowledge graph.

For example, the electronic apparatus may extract a negative sample with a label of 0 by replacing one of a head entity (h), a relation information (r), and a tail entity (t) among the triple structure (h, r, t) with a different value. In addition, the electronic apparatus may extract a triple structure observed in the knowledge graph as a positive sample with a label of 1.

The electronic apparatus may train the GNN based on the knowledge graph using the negative sample with a label of 0 and the positive sample with a label of 1.

In an example, a KGE model may be trained through the above-described embedding model (e.g., the TransE model). The KGE model may have respective entities, relation information, and a score function corresponding thereto. A KGE value learned based on such a score function may be used as an initial value of another task, or may be added to a main first loss and simultaneously learned through multi-task learning, improving the performance of the GNN.

The electronic apparatus may train the prediction neural network such that the relation information reflects the tendency of changes in physical property values among nodes according to a change in a structural characteristic of a material corresponding to each of the nodes of the knowledge graph.

The electronic apparatus may train the prediction neural network based on the first loss (LMolCLR) based on MolCLR and the second loss (LKG) weighted by a weight as expressed by Equation 4 below.

$${ℒ}_{total} = {ℒ}_{MolCLR} + {w * {ℒ}_{KG}}_{weight} \qquad Equation 4$$

Thus, the electronic apparatus may train the prediction neural network based on relation information (e.g., ΔProperty) embedded in the vector space.

The electronic apparatus may train the prediction neural network (e.g., the prediction MLP 310 and the prediction MLP 350) to respectively predict a characteristic corresponding to each of the materials based on the tendency of changes in physical property values predicted by the GNN. Accordingly, during an inference operation, characteristics of a target material may be predicted by providing a molecular representation of the target material to either of the trained prediction MLPs 310 and 350 during the inference operation.

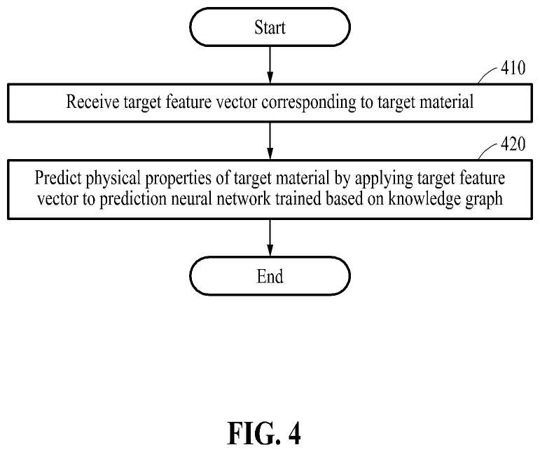

FIG. 4 illustrates an example method of predicting physical properties using a trained neural network according to one or more embodiments.

Referring to FIG. 4, an electronic apparatus (or system) for predicting physical properties may predict the physical properties of a target material through operations 410 and 420.

In operation 410, the electronic apparatus may receive a target feature vector corresponding to the target material. The target feature vector may be, for example, the above-described molecular representation h, but examples are not limited thereto.

In operation 420, the electronic apparatus may predict the physical properties of the target material by applying the target feature vector received in operation 410 to a prediction neural network trained based on a knowledge graph according to any of the training operations discussed herein. For example, as illustrated in FIGS. 2A and/or 2B, the knowledge graph may have feature vectors representing the structures of materials as nodes and have relation information between the materials as an edge. The feature vectors may correspond to material representations including a material structure or molecular structure of each of the materials. The materials may be understood to encompass both organic matters (organic materials) and inorganic matters (inorganic materials). The relation information may include changes in physical property values indicating changes in characteristics according to changes in structures among the materials. The changes in physical property values, for example, may be represented in the form of consecutive numbers, and may include multi-dimensional edges according to the types of materials.

The knowledge graph may have a triple structure including a head entity and a tail entity corresponding to nodes, and relation information related to changes in physical property values between the head entity and the tail entity. The head entity, the tail entity, and the relation information may all be represented as vectors. Alternatively, some of the head entity, the tail entity, and the relation information may be represented as matrices while the others may be represented as vectors.

The prediction neural network may be trained to predict physical properties corresponding to the distances between the nodes in a vector space. The vector space may define operations of addition and scalar product and may refer to a set (space) that satisfies predetermined conditions for these operations. In an example, the vector space may correspond to a space in which elements may be added together or increased or decreased by a given multiple. In an example, the vector space may be referred to as a “linear space”, which may include, for example, a Euclidean space, but examples are not limited thereto.

As described above, the prediction neural network may be trained based on the first loss (LMolCLR) based on MolCLR and the second loss (LKG) corresponding to the knowledge graph. The second loss may be a value (w×LKG) weighted by a weight (w).

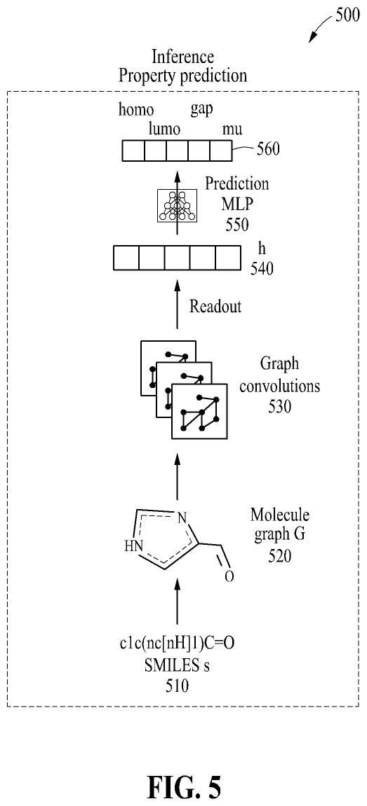

FIG. 5 illustrates an example inference operation using a GNN, which has been trained based on a molecule-based knowledge graph, according to one or more embodiments. FIG. 5 is a diagram 500 illustrating a process in which an electronic apparatus (or system) predicts physical properties 560 corresponding to a SMILES s 510 using a prediction neural network 550.

When the SMILES s 510 is input, the electronic apparatus may represent the SMILES s 510 as a molecular graph G 520. The electronic apparatus may obtain a molecular representation h 540 by passing the molecular graph G 520 through a neural network 530 and then outputting (or performing a readout with respect to) results of the neural network 530.

The electronic apparatus may predict the physical properties 560 (e.g., HOMO label, LUMO label, GAP, dipole moment (μ), etc.) corresponding to the SMILES s 510 by applying the molecular representation h 540 to a prediction neural network 550 trained through the above-described process.

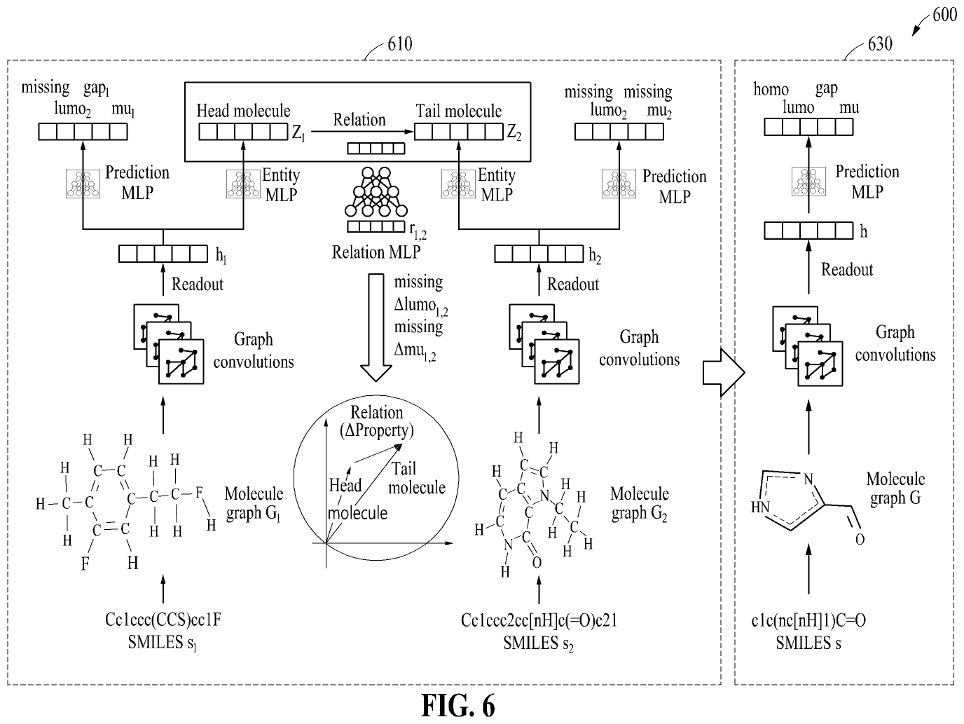

FIG. 6 illustrates an example method of training a GNN when some data for application to the GNN is missing (e.g., not available or not necessarily used) in training data according to one or more embodiments. FIG. 6 is a diagram 600 illustrating a training process 610 and an inference process 630 in the case of sparse data, where some data applied to the GNN is missing.

An open dataset, which may be the most common type of dataset used in machine learning, typically has all data filled in without blanks, but a sparse dataset structure in which some or a majority of the data is missing may also/alternatively be used to perform the machine learning training.

By embedding nodes in a vector space through relation information between nodes, it may be possible to achieve high prediction performance even in cases where some training data is missing (e.g., not available), with relatively little performance degradation.

Even when a portion of relation information (e.g., Δlumo1,2 and Δmu1,2) does not exist in an arbitrary triple structure of a knowledge graph, an electronic apparatus (or system) may predict the missing relation information.

The above-described TransE is trained to satisfy the condition h+r=t and may thus derive r=t−h. Accordingly, the electronic apparatus may calculate a relation vector (r) by finding a vector that is most similar to the result of subtracting a head entity vector (h) from a tail entity vector (t).

An embedding model of the knowledge graph may provide effective performance compared to a simply randomly initialized vector because the embedding model learns sufficient information about the (molecular) structure of the knowledge graph(s) during the machine learning training process. Accordingly, the embedding model may be utilized for multi-task learning as well.

For example, the electronic apparatus may train the prediction neural network using not only a QA loss but also the loss of the embedding model of the knowledge graph when assuming a QA model using the knowledge graph.

By utilizing relation information to train the illustrated prediction neural network, the training apparatus may exhibit robustness against a sparse dataset compared to using only feature vectors corresponding to nodes.

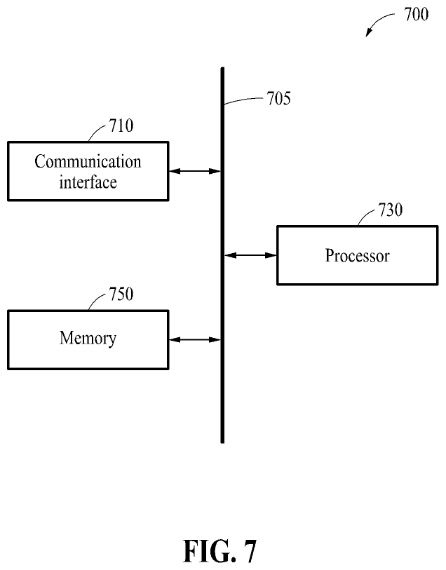

FIG. 7 illustrates an example electronic apparatus (or system) according to one or more embodiments. Referring to FIG. 7, an electronic apparatus (or system) 700 may include a communication interface 710, a processor 730, and a memory 750. The communication interface 710, the processor 730, and the memory 750 may communicate with each other via a communication bus 705.

The communication interface 710 may receive a target feature vector corresponding to a target material. Alternatively, the processor 730 may obtain the target feature vector from the memory 750.

The processor 730 may predict physical properties of the target material by applying the target feature vector to a prediction machine learning model, e.g., a prediction neural network, trained based on knowledge graph(s) according to any one or any combination of the training operations described herein. The knowledge graph may have feature vectors representing materials as nodes and have relation information between the materials as an edge. As a non-limiting example, the relation information may be in a vector or matrix form. As described above with respect to FIGS. 1-6, the prediction machine learning model may be trained to predict physical properties corresponding to a distance between the nodes in a vector space.

The memory 750 may store a various pieces of information generated in the processing processes of the processor 730 described above. In addition, the memory 750 may store various types of data and programs. The memory 750 may include a volatile memory or a non-volatile memory. The memory 750 may include a large-capacity storage medium such as a hard disk to store various types of data.

The processor 730 may perform any one or any combination of operations or methods, e.g., as respective algorithms, described above with reference to FIGS. 1 to 6 through execution of corresponding instructions stored in the memory 750. For example, the processor 730 is a physical structure hardware data processing device having circuitry configured to execute instructions (e.g., as code or programs) stored in the memory 750. The processor 730 may be representative of, for example, a central processing unit (CPU), a graphics processing unit (GPU), and/or a neural network processing unit (NPU). In an example, the electronic apparatus 700 may be hardware that includes, for example, a microprocessor, a CPU, a processor core, a multi-core processor, a multiprocessor, an application-specific integrated circuit (ASIC), and a field-programmable gate array (FPGA), as non-limiting examples.

Accordingly, the processor 730 may execute instructions and control and/or configure the electronic apparatus 700, including the processor 730, to perform one or more, or any combination of, the operations and methods described herein based on those executed instructions.

The processors, memory, and communication interface described herein, including corresponding and other descriptions herein with respect to respect to FIGS. 1-7, are implemented by or representative of hardware components. As described above, or in addition to the descriptions above, examples of hardware components that may be used to perform the operations described in this application may include controllers, sensors, generators, drivers, memories, comparators, arithmetic logic units, adders, subtractors, multipliers, dividers, integrators, and any other electronic components configured to perform the operations described in this application. In other examples, one or more of the hardware components that perform the operations described in this application are implemented by computing hardware, for example, by one or more processors or computers. A processor or computer may be implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices that is configured to respond to and execute instructions in a defined manner to achieve a desired result. In one example, a processor or computer includes, or is connected to, one or more memories storing instructions or software that are executed by the processor or computer. Hardware components implemented by a processor or computer may execute instructions or software, such as an operating system (OS) and one or more software applications that run on the OS, to perform the operations described in this application. The hardware components may also access, manipulate, process, create, and store data in response to execution of the instructions or software. For simplicity, the singular term “processor” or “computer” may be used in the description of the examples described in this application, but in other examples multiple processors or computers may be used, or a processor or computer may include multiple processing elements, or multiple types of processing elements, or both. For example, a single hardware component or two or more hardware components may be implemented by a single processor, or two or more processors, or a processor and a controller. One or more hardware components may be implemented by one or more processors, or a processor and a controller, and one or more other hardware components may be implemented by one or more other processors, or another processor and another controller. One or more processors, or a processor and a controller, may implement a single hardware component, or two or more hardware components. As described above, or in addition to the descriptions above, example hardware components may have any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

The methods illustrated in, and discussed with respect to, FIGS. 1-7 that perform the operations described in this application are performed by computing hardware, for example, by one or more processors or computers, implemented as described above implementing instructions (e.g., computer or processor/processing device readable instructions) or software to perform the operations described in this application that are performed by the methods. For example, a single operation or two or more operations may be performed by a single processor, or two or more processors, or a processor and a controller. One or more operations may be performed by one or more processors, or a processor and a controller, and one or more other operations may be performed by one or more other processors, or another processor and another controller. One or more processors, or a processor and a controller, may perform a single operation, or two or more operations.

Instructions or software to control computing hardware, for example, one or more processors or computers, to implement the hardware components and perform the methods as described above may be written as computer programs, code segments, other instructions or any combination thereof, for individually or collectively instructing or configuring the one or more processors or computers to operate as a machine or special-purpose computer to perform the operations that are performed by the hardware components and the methods as described above. In one example, the instructions or software include machine code that is directly executed by the one or more processors or computers, such as machine code produced by a compiler. In another example, the instructions or software includes higher-level code that is executed by the one or more processors or computer using an interpreter. The instructions or software may be written using any programming language based on the block diagrams and the flow charts illustrated in the drawings and the corresponding descriptions herein, which disclose algorithms for performing the operations that are performed by the hardware components and the methods as described above.

The instructions or software to control computing hardware, for example, one or more processors or computers, to implement the hardware components and perform the methods as described above, and any associated data, data files, and data structures, may be recorded, stored, or fixed in or on one or more non-transitory computer-readable storage media, and thus, not a signal per se. As described above, or in addition to the descriptions above, examples of a non-transitory computer-readable storage medium include one or more of any of read-only memory (ROM), random-access programmable read only memory (PROM), electrically erasable programmable read-only memory (EEPROM), random-access memory (RAM), dynamic random access memory (DRAM), static random access memory (SRAM), flash memory, non-volatile memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, blue-ray or optical disk storage, hard disk drive (HDD), solid state drive (SSD), flash memory, a card type memory such as multimedia card micro or a card (for example, secure digital (SD) or extreme digital (XD)), magnetic tapes, floppy disks, magneto-optical data storage devices, optical data storage devices, hard disks, solid-state disks, and/or any other device that is configured to store the instructions or software and any associated data, data files, and data structures in a non-transitory manner and provide the instructions or software and any associated data, data files, and data structures to one or more processors or computers so that the one or more processors or computers can execute the instructions. In one example, the instructions or software and any associated data, data files, and data structures are distributed over network-coupled computer systems so that the instructions and software and any associated data, data files, and data structures are stored, accessed, and executed in a distributed fashion by the one or more processors or computers.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents.

Therefore, in addition to the above and all drawing disclosures, the scope of the disclosure is also inclusive of the claims and their equivalents, i.e., all variations within the scope of the claims and their equivalents are to be construed as being included in the disclosure.

What is claimed is:

1. A processor-implemented method, the method comprising:
predicting physical properties of a target material using a machine learning model provided an input that is based on a target feature vector, where the target feature vector corresponds to the target material,
wherein the machine learning model is configured to predict the physical properties of the target material based on a multi-dimensional space that is dependent on feature vectors representing respective structures of materials and relation information between the materials.

2. The method of claim 1, wherein each of the feature vectors corresponds to a node of a knowledge graph as a material representation of a material structure or molecular structure of a corresponding material, and the relation information corresponds to an edge of the knowledge graph.

3. The method of claim 1, wherein, with respect to the multi-dimensional space, the relation information corresponds to changes in physical property values indicating changes in characteristics according to structural changes among the materials.

4. The method of claim 3,
wherein each of the feature vectors corresponds to a node of a knowledge graph, and the relation information corresponds to an edge of the knowledge graph, and
wherein the changes in physical property values are represented in the knowledge graph in a form of consecutive numbers and configured with multi-dimensional edges based on the materials.

5. The method of claim 1,
wherein each of the feature vectors corresponds to a node of a knowledge graph, and the relation information corresponds to an edge of the knowledge graph, and
wherein the knowledge graph has a triple structure comprising a head entity and a tail entity respectively corresponding to the nodes, and the relation information is related to a change in a physical property value between the head entity and the tail entity.

6. The method of claim 1,
wherein each of the feature vectors corresponds to a node of a knowledge graph, and the relation information corresponds to an edge of the knowledge graph, and
wherein the prediction machine learning model is trained to predict physical properties corresponding to a distance between the nodes in a vector space, as the multi-dimensional space.

7. The method of claim 1,
wherein each of the feature vectors corresponds to a node of a knowledge graph, and the relation information corresponds to an edge of the knowledge graph,
wherein the multi-dimensional space is a vector space embedded with the relation information, and
wherein the method further comprises training the machine learning model based on a first loss based on molecular contrastive learning of representations (MolCLR) and a weighted second loss corresponding to the knowledge graph.

8. A processor-implemented method, the method comprising:
generating a knowledge graph having, as respective nodes of the knowledge graph, a head entity corresponding to a first feature vector representing a structure of a first material and a tail entity corresponding to a second feature vector representing a structure of a second material, and having relation information between the first material and the second material as an edge of the knowledge graph;
embedding the knowledge graph into a vector space; and
training a prediction machine learning model based on the relation information embedded in the vector space.

9. The method of claim 8,
wherein the first feature vector corresponds to a first molecular representation representing a molecular structure of the first material,
wherein the second feature vector corresponds to a second molecular representation representing a molecular structure of the second material, and
wherein the first feature vector and the second feature vector are calculated based on molecular contrastive learning of representations (MolCLR).

10. The method of claim 9, wherein the first molecular representation and the second molecular representation respectively correspond to different but correlated molecular graphs.

11. The method of claim 8, wherein the relation information comprises changes in physical property values indicating changes in characteristics according to structural changes among the first and second materials.

12. The method of claim 8, wherein the embedding of the knowledge graph into the vector space comprises embedding the knowledge graph into the vector space using an embedding model configured to perform embedding based on a distance between a result of adding the relation information to the head entity and the tail entity.

13. The method of claim 8,
wherein the training of the prediction machine learning model is based on respective outputs of a first neural network configured to output a first latent vector corresponding to a molecular structure of the head entity, and a second neural network configured to output a second latent vector corresponding to a molecular structure of the tail entity, and
wherein the training of the prediction machine learning model includes training, dependent on the respective outputs, a relation neural network configured to enable an embedding vector corresponding to a change in a physical property value between the first latent vector and the second latent vector to be matched to the relation information.

14. The method of claim 8, wherein the training of the prediction machine learning model comprises training the prediction machine learning model by combining the relation information with a respective unique structural characteristic of each of the first material and the second material.

15. The method of claim 8, wherein the training of the prediction machine learning model comprises training the prediction machine learning model to reflect a tendency of changes in physical property values among the respective nodes according to respective changes in structural characteristics of the first and second materials.

16. The method of claim 8,
wherein the training of the prediction machine learning model comprises:
calculating a second loss corresponding to the knowledge graph based on a distance between the tail entity and the head entity translated by the relation information in the vector space; and
training the prediction machine learning model based on a weighting of the second loss.

17. The method of claim 16, wherein the calculating of the second loss comprises calculating the second loss corresponding to the knowledge graph as a negative margin loss calculated from the embedding of the knowledge graph.

18. The method of claim 16, wherein the training of the prediction machine learning model based on the weighted second loss comprises training the prediction machine learning model based on a first loss based on molecular contrastive learning of representations (MolCLR) and the weighted second loss.

19. The method of claim 8, wherein the method further comprises predicting physical properties of a target material using the trained prediction machine learning model provided an input that is based on a target feature vector, where the target feature vector corresponds to the target material.

20. An electronic apparatus, the apparatus comprising:
one or more processors configured to execute instructions; and
a memory storing the instructions, which when executed by the one or more processors, configures the one or more processors to predict physical properties of a target material using a machine learning model provided an input that is based on a target feature vector, where the target feature vector corresponds to the target material,
wherein the machine learning model is configured to predict the physical properties of the target material based on a multi-dimensional space that is dependent on feature vectors representing respective structures of materials and relation information between the materials.
